(19) United States
(12) Patent Application Publication
(10) Pub. No.: US 2012/0231480 A1
(43) Pub. Date: Sep. 13, 2012

(54) MATERIALS AND METHODS FOR THE DIFFERENTIAL DIAGNOSIS OF PACREATIC LESIONS

(75) Inventors: C. Max Schmidt, Zionsville, IN (US); Michele Yip-Schneider, Carmel, IN (US)

(73) Assignee: Indiana University Research & Technology Corporation, Indianapolis, IN (US)

(21) Appl. No.: 13/392,417

(22) PCT Filed: Aug. 27, 2010
(86) PCT No.: PCT/US10/47017

§ 371 Date: May 30, 2012

Related U.S. Application Data
(60) Provisional application No. 61/237,346, filed on Aug. 27, 2009.

(51) Int. Cl. G01N 33/574

(52) U.S. Cl. 435/7.92

(57) ABSTRACT

Levels of VEGF-A, VEGF-C and PGE₂ were measured in patient groups presenting with different types of pancreatic lesions. Pancreatic fluids that exhibited high levels of VEGF-A and relative low levels of VEGF-C correlated well with a diagnosis of serous cystadenoma. Pancreatic fluids collected from cysts in patients that did not have a clinical diagnosis of pancreatic cancer and that exhibited relatively low levels of VEGF-A correlated with a diagnosis of intraductal papillary mucinous neo-plasms (IPMN). Furthermore, the level of PGE₂ increased with dysplastic stage. Contacting pancreatic fluid with reagents that selectively bind to VEGF-A, VEGF-C and PGE₂ provide a useful diagnostic tool for identifying patients with these benign, pre-malignant or malignant lesions of the pancreas.

### PRIORITY CLAIM

This application claims the benefit of U.S. Provisional Patent Application No. 61/237,346 filed on Aug. 27, 2010, and incorporated herein by reference in its entirety.

### STATEMENT OF GOVERNMENTAL RIGHTS

Part of the work during the development of this invention was made with support provided by the U.S. Federal Government from the National Institute of Health under grant number NIH IR03CA112629-01A.

### BACKGROUND

Pancreatic cancer is among the most lethal forms of cancer ranking as the fourth leading cause of death among American men and women. According to statistics released on May 10, 2009 by the National Institute of Health, less than five percent of patients diagnosed with the disease are still alive five years after being diagnosed with the disease. Reasons for this cancer's high rate of morbidity include the dearth of effective treatments for the disease and the fact that most patients are asymptomatic from the disease until it is well advanced. Pancreatic cancer, like most cancers, is best treated in its early pre-metastatic stages.

In addition to its high morbidity, pancreatic cancer is especially troubling because the number of individuals with risk factors for developing the disease, which include smoking, obesity and diabetes, is increasing. According to the World Health Organization, worldwide over one billion people are overweight and over 300 million of these people are obese. Moreover, the number of overweight and obese people is growing in most countries including the world's most populous countries, China and India, as people in these countries adopt more sedentary life styles and calorie rich diets.

In the United States alone, over 30 percent of the adult population is either overweight or obese. And the rates of obesity in American children are increasing at an alarming rate. According to the Center for Disease Control as of the year 2006, the percentage of obese children aged 6 to 11 more than doubled, increasing from 6.5% in 1980 to 17.0% in 2006. The rate of obesity among adolescents aged 12 to 19 more than tripled, increasing from 5% to 17.6%. See, Ogden C L, et al., “High Body Mass Index for Age Among US Children and Adolescents,” 2003-2006, JAMA, 2008; 299(20):2401-2405. A study published in the Journal of the American Medical Society reported that obese young adults have an increased risk of developing pancreatic cancer. See e.g., Donghui Li, PhD; et al., JAMA., 301[24]:2553-2562. Given the seriousness of pancreatic cancer, the difficulty in diagnosing the disease and the increasing number of people with a heightened risk for developing the disease, there is great need for additional materials and methods for identifying individuals with pre-malignant as well as malignant pancreatic lesions. Some aspects and embodiments of the present invention address this need.

### SUMMARY

Some embodiments include methods for helping to distinguish between benign, pre-malignant or malignant pancreatic lesions, comprising the steps of: contacting a sample of pancreatic fluid with at least one reagent that selectively binds to VEGF-A and/or VEGF-C and measuring the level of VEGF-A and/or VEGF-C in the sample and then comparing the level of VEGF-A and/or VEGF-C in the sample to levels of VEGF-A and/or VEGF-C measured in samples obtained from patients that are known to have benign, pre-malignant or malignant pancreatic lesions. In some embodiments the levels of both VEGF-A and VEGF-C are measured in the same sample and combined to a create a value that is in turn compared to values of VEGF measured in samples recovered from patients with a specific pancreatic lesion diagnosis. In some embodiments levels of VEGF-A measured in a given sample are compared to levels of VEGF-A measured for a number of samples. In some embodiments levels of VEGF-C measured in a given sample are compared to levels of VEGF-C measured for a number of samples. Still other embodiments, may further include the steps of: contacting a sample of pancreatic fluid with at least one reagent that binds to PGE₂ in the sample and measuring the amount of PGE₂ in the sample; and comparing the level of PGE₂ in the sample to the level of PGE₂ in samples from patients known to have benign, pre-malignant or malignant pancreatic lesions.

Some embodiments include the step of determining that a patient has either serous cystadenoma or cystic adenocarcinma NOS (stage IV (metastatic) mucinous adenocarcinoma) if the level of VEGF-A measured in the patient's pancreatic fluid is greater than about 7,500 pg ml⁻¹. Some embodiments include the further step of measuring the level of VEGF-C in a sample of pancreatic fluid which has a level of VEGF-A of greater than about 7,500 pg ml⁻¹.

Some embodiments include the step of diagnosing the patient as having serous cystadenoma if the level of VEGF-C measured in the patient's pancreatic fluid is greater than about 750 pg ml⁻¹ and the level of VEGF-A in the patient's pancreatic fluid is greater than about 7,500 pg ml⁻¹.

Some embodiments include the step of diagnosing the patient as having cystic adenocarcinoma NOS (stage IV (metastatic) mucinous adenocarcinoma) if the level of VEGF-C measured in the patient's pancreatic fluid is less than about 750 pg ml⁻¹ and the level of VEGF-A in the patient's pancreatic fluid is greater than about 7,500 pg ml⁻¹.

Some embodiments include the step of diagnosing a patient as having about a 9 in 10 chance of having IPMN moderate, high, invasive or pancreatic cancer and a 1 in 10 chance of having a pseudocyst if the patient has at least one pancreatic cyst and the patient's pancreatic fluid has a level of VEGF-A of less than about 7,500 pg ml⁻¹ and a level of PGE₂ of greater than about 1,250 pg ml⁻¹.

Some embodiments include the step of diagnosing a patient as having IPMN adenoma or a mucinous cystadenoma if the patient has at least one pancreatic cyst and the patient's pancreatic fluid has a level of VEGF-A of less than about 7,500 pg ml⁻¹ and a level of PGE₂ of less than about 1,250 pg ml⁻¹.

In some embodiments a diagnosis may be based on determining the levels of PGE₂ and VEGF-A and VEGF-C in at least one sample obtained from the same patient and comparing these levels to levels of PGE₂ and VEGF-A and/or VEGF-C measured in patients that have already been unambiguously diagnosed with benign, pre-malignant or malignant pancreatic lesions.

Still other embodiments include a kit for analyzing pancreatic secretions, comprising: at least one reagent that preferentially binds to VEGF-A and/or VEGF-C in a sample of pancreatic fluid. In other embodiments, the kit further includes at least one additional reagent that preferentially binds to PGF₂, in a sample of pancreatic fluid. Some embodiments these kits include, buffer, anti-oxidants, anti-microbial compound and the like.

### BRIEF DESCRIPTION OF THE FIGURES

FIG. 1. Bar graph illustrating the level of VEGF measured in samples of pancreatic fluid collected from patients with various types of pancreatic lesions, including data from sample 117 with uncertain diagnosis (IPMN invasive or mucinous cystadenocarcinoma). Bars, mean (pg ml⁻¹) +/−SEM; p<0.001 for serous cysts versus all of the other groups identified.

FIG. 2. Graph of individual data points illustrating the level of VEGF measured in samples of pancreatic fluid collected from patients with various types of pancreatic lesions, including data from sample 117, with uncertain diagnosis (IPMN invasive or mucinous cystadenocarcinoma), as indicated.

FIG. 3. Graph of individual data points illustrating the level of VEGF measured in samples of pancreatic fluid collected from patients with various types of pancreatic lesions, excluding data from sample 117, with uncertain diagnosis.

FIG. 4. Bar graph illustrating the level of VEGF measured in samples of serum collected from patients with various types of pancreatic lesions. Bars, mean (pg ml⁻¹) +/− SEM.

FIG. 5. Bar graph illustrating the level of VEGF measured in samples of bile collected from patients with various types of pancreatic lesions. Bars, mean (pg ml⁻¹) +/− SEM.

FIG. 6. Bar graph illustrating the level of VEGF measured in different fluids collected from individual patients diagnosed with the indicated pancreatic lesions. Bars, mean (pg ml⁻¹) +/− SEM.

FIG. 7. Table summarizing the immunohistochemical analysis of VEGF-A and VEGF-Receptor-2 expression in patients diagnosed with different types of pancreatic lesions. The levels of VEGF were measured by ELISA in the corresponding patients are also shown.

FIG. 8A. Photomicrograph of normal pancreatic tissue stained with an antibody specific for VEGF-A, magnified 400 times.

FIG. 8B. Photomicrograph of pancreatic tissue from a patient diagnosed with mucinous cystadenoma stained with an antibody specific for VEGF-A, magnified 400 times.

FIG. 8C. Photomicrograph of pancreatic tissue from a patient diagnosed with serous cystadenoma stained with an antibody specific for VEGF-A, magnified 400 times.

FIG. 8D. Photomicrograph of pancreatic tissue from a patient diagnosed with IPMN stained with an antibody specific for VEGF-A, magnified 400 times.

FIG. 9. Graph of individual data points illustrating the level of PGE₂ pg ml⁻¹ measured in samples of pancreatic fluid collected from patients with various types of pancreatic lesions.

FIG. 10. Graph of individual data points illustrating the level of PGE₂ measured in samples of pancreatic fluid collected from patients with various types of pancreatic lesions including data from patients with benign pseudocysts (false cysts).

FIG. 11. Graph of individual data points illustrating the level of VEGF-C measured in samples of pancreatic fluid collected from patients with various types of pancreatic lesions including data from patients with benign pseudocysts (false cysts).

FIG. 12. Bar graph, complete with error bars, of data illustrating the level of VEGF-C measured in samples of pancreatic fluid collected from patients with various types of pancreatic lesions including data from patients with benign pseudocysts (false cysts).

FIG. 13. A flow chart illustrating some of the steps in the process of using the amount of VEGF-A and/or VEGF-C singled-out from a sample of fluid to distinguish between different types of pancreatic lesions.

### DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates.

The term, ‘about’ as used herein with regard to numeric values means plus or minus 10 percent, e.g. about 100 pg ml⁻¹ encompasses a range of values from 90 pg ml⁻¹ to 110 pg ml⁻¹.

Currently, serous cystadenoma are identified by cross sectional imaging (CT or MRI) which demonstrates classically a central stellate scar within the fluid density lesion. These lesions are often microcystic and lack a ductal connection. EUS-FNA of the cyst fluid demonstrates no evidence of mucin and low CEA levels.

Currently, cystic adenocarconoma NOS (stage IV (metastatic) mucinous adenocarcinoma is diagnosed by biopsy of a liver lesion or peritoneal implant by the surgeon. Histology demonstrates adenocarcinoma with mucinous features.

Currently, pseudocysts are diagnosed by clinical history and physical exam which includes symptoms and findings associated with prior or ongoing acute pancreatitis. Cross sectional imaging (MRI, CT) demonstrates well circumscribed round fluid density lesion within or adjacent to the pancreatic parenchyma of varying sizes, with or without evidence of debris or hemorrhage within the cyst. Ductal continuity is typically visible on imaging. If sampled, these lesions develop high levels of amylase consistent with pancreatic juice.

Currently, intraductal papillary mucinous neoplasms (IPMN) are diagnosed using initial cross sectional imaging (optimally MRCP) which demonstrates a single or multiple fluid density lesions in the pancreatic gland involving either the main or secondary pancreatic ducts. These lesions are often multifocal and multicentric. Fine needle aspiration under endoscopic ultrasound (EUS) guidance of the cyst fluid generally reveals mucin, with evidence of mucinous epithelium with varying degrees of dysplasia on cytologic examination. Elevated CEA and amylase levels are consistent with IPMN.

Currently, mucinous cystic neoplasms (MCN) are diagnosed by cross sectional imaging (CT or MRI). These lesions are usually unifocal and macrocystic, may have mural calcifications, and lack a ductal connection. EUS-FNA demonstrates mucinous lesions with elevated CEA level, but low amylase (due to the lack of ductal connetion).

The histologic grade of IPMN is based on the highest level of dysplasia present in the lesion. This can be determined by cytologic sample obtained from cyst fluid or wall during EUS-FNA or core biopsy. Criteria for cytologic atypia included at least 1 of the following: increased nuclear-cytoplasmic ratio, increased nuclear size, nuclear crowding, or hyperchromasia. Ultimately this is determined via pathologic analysis of a permanently prepared surgical pancreas specimen. The histologic grades are defined in the following ways: adenoma (dilated pancreatic duct lined by mucinous epithelium, with ≦1 criteria for low-grade dysplasia; also called duct ectasia), moderate (≧2 of the following criteria: epithelial tufuting, nuclear pseudostratification, nuclear atypia, and mitotic figures; also called borderline), high-grade dysplasia (cribiform or solid growth usually associated with high grade nuclear atypical; also called non-invasive intraductal carcinoma or carcinoma in situ), and invasive (disruption of the ductal basement membrane and extension of dysplastic cells into the pancreatic tissue with or without lymphovascular invasion.

Additional information on the identification, categorization and characterization of pancreatic lesions including pancreatic cysts as it is currently practice in the surgical arts can be found in treatises such as, Current Surgical Therapy, edited by John L. Cameron (9^{th} ed, 1397 pp, Philadelphia, Pa., Mosby/Elsevier, 2008), and similar texts, reviews, manuals and papers known in the art.

Vascular endothelia growth factor A ('VEGF-A′) is a protein identified in humans and encoded by the VEGFA gene. Mattei, M. G.; Borg, J. P.; Rosnet, O.; Marine D.; Birnbaum D. (February 1996). “Assignment of vascular endothelial growth factor (VEGF) and placenta growth factor (PLGF) genes to human chromosome 6p12-p21 and 14q24-q31 regions, respectively”. Genomics 32 (1): 168-9.

Vascular endothelia growth factor C ('VEGF-C′) is a protein identified in humans and encoded by the VEGFC gene. Joukov, V.; Pajusola, K.; Kaipainen, A.; Chilov, D.; Lahtinen, I.; Kukk, E.; Saksela, O.; Kalkkinen, N.; and Alitalo, K. (1996). “A novel vascular endothelial growth factor, VEGF-C, is a ligand for the Flt4 (VEGFR-3) and KDR (VEGFR-2) receptor tyrosine kinases.” EMBO J. 15 (2): 290-98.

Prostaglandin E₂ (7-[3-hydroxy-2-(3-hydroxyoct-1-enyl)-5-oxo-cyclopentyl] hept-5-enoic acid) (‘PGE₂’). Schmidt, Max, C., et al., J. Gastrointest. Surg. (2008) 12:243-249.

Pancreatic cancer is the fourth leading cause of cancer-related deaths, with mortality nearly equal to incidence. Distinct types of premalignant pancreatic lesions have been categorized as pancreatic intraepithelial neoplasias (PanIN), intraductal papillary mucinous neoplasms (IPMN), or mucinous cystic neoplasms (MCN). These lesions have the potential to give rise to invasive adenocarcinoma. Cystic lesions of the pancreas are being diagnosed in increasing numbers due, at least in part, to the use of high resolution imaging. Cystic lesions include IPMN, MCN and serous cystadenoma (SCA). Unlike IPMN and MCN, SCA is a benign neoplastic cyst of the pancreas which has an extremely low risk of malignant transformation. In general, although symptomatic SCAs require surgery, asymptomatic neoplasms currently do not. For optimal treatment and to avoid unnecessary surgery, it is clinically important to distinguish between low-risk pancreatic cysts (SCA or pseudocyst) which are generally not resected and high risk neoplastic mucinous cysts (MCN and IPMN) which are often resected. Identification of suitable biomarkers will greatly facilitate diagnosis, prevention and/or treatment. Theoretically, if possible it may be advantageous to isolate such biomarkers from tissue, serum, pancreatic or other bodily fluids. Existing serum biomarkers for pancreatic cancer such as CEA (carcinoembryonic antigen) and CA 19-9 (carbohydrate 19-9 antigen) have been identified. Unfortunately, the identification of these particular biomarkers in various samples have been shown to be dispositive for diagnosing pancreatic cancers due, perhaps at least in part, to their low sensitivity and specificity. Tissue and serum levels of the angiogenic factor vascular endothelial growth factor (VEGF) have been shown to be elevated in pancreatic cancer patients. While pancreatic cyst fluid from SCAs and pseudocysts typically exhibit low CEA levels; in contrast, mucinous lesions of the pancreas show elevated CEA levels. However, there are exceptions to these trends.

If they exist, the identification of diagnostic biomarkers for pancreatic cancers and pre-cancerous disorders may enhance the potential for biomarker screening based assays to distinguish between different types of pancreatic lesions. Ultimately, a screen that incorporates at least some of any new biomarkers should facilitate earlier detection and diagnosis of disease, thereby increasing the likelihood of successful chemoprevention or chemotherapy for this deadly disease. The instant disclosure includes identification of new biomarkers and in some of the embodiments methods of using these new biomarkers sometimes in combination with other biomarkers to better distinguish between precancerous and otherwise benign pancreatic lesions.

PGE₂ levels were determined in patient pancreatic fluid collected at the time of endoscopy or operation. And mean PGE₂ levels were shown to be significantly higher in IPMNs than in MCNs. Furthermore, the level of PGE₂ increased with dysplastic stage. Thus, this work suggests that PGE₂ level may help differentiate IPMN from MCN in patients with known mucinous lesions and that it may also be an indicator of malignant progression of IPMNs in some patients. See, Schmidt, Max, C., et al., J. Gastrointest. Surg. (2008) 12:243-249.

Levels of prostaglandin E-2 (PGE₂) measured in pancreatic fluids that also have levels of VEGF-A in excess of 7,500 pg ml⁻¹ can be used to distinguish between cysts that are IPMN (moderate, high grade, or invasive), pancreatic cancers, or pseudocysts and cysts that are IPMN adenoma or mucinous cystadenoma (mucinous cystic neoplasms (MCN)) provided that samples are collected from patients that do not have an obstructed main pancreatic duct.

As disclosed herein, contacting samples of pancreatic fluid with reagents that selectively bind to vascular endothelial growth factor (VEGF-A) can be used to measure the levels of VEGF-A in the sample and this measurement can be used to distinguish between serous cystadenomas (SCA) and pre-cancerous pancreatic cysts. Levels of VEGF-A measured in SCA are significantly higher than VEGF-A levels measured in pseudocytes or in IPMN adenoma/moderate grade or IPMN high grade/invasive, mucinous cystadenoma, or pancreatic ductile adenocarcinoma pancreatic lesions. Thus, PGE₂ and VEGF-A and VEGF-C in contact with the proper reagents such as antibodies to the respective gene products may serve as biomarkers for the diagnosis, and/or characterization of pancreatic lesions. And when used together in a panel, these biomarkers are at least as diagnostic in terms of both selectivity and specificity as the biomarkers currently used and/or identified in the literature.

### Experimental

1. Measuring VEGF in Healthy Volunteers and Volunteers Diagnosed with Pancreatic Lesions.

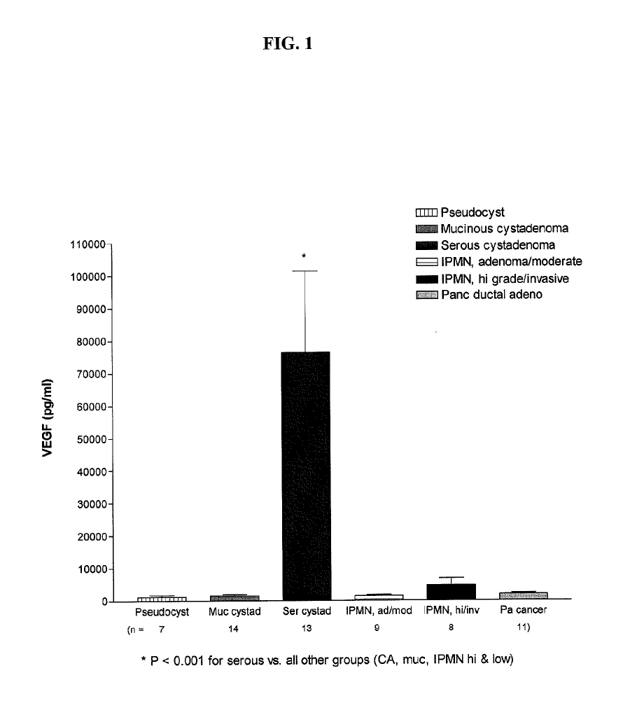

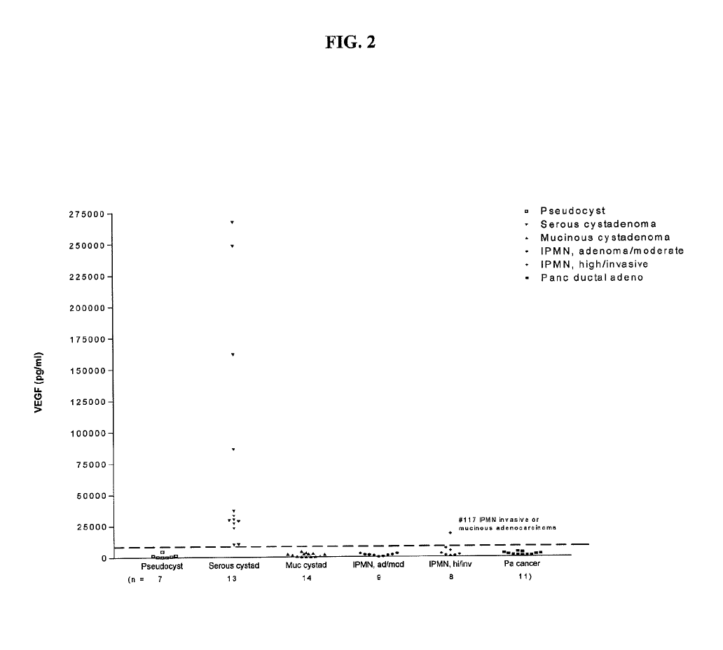

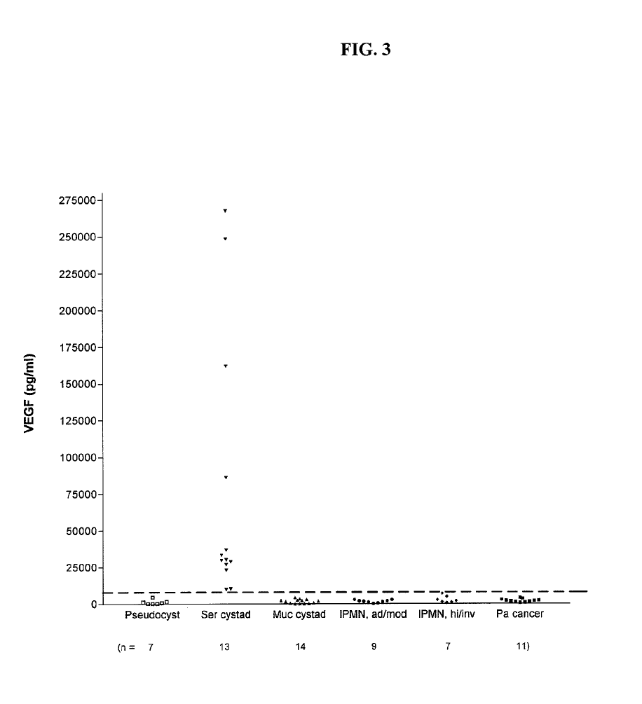

As a requirement for admission to this study, all patients signed informed consent forms for collection of pancreatic cyst and/or ductile fluid at the time endoscopy (EUS or ERCP) or operation (OR) at Indiana University Hospital per the Indiana University Pancreatic Tissue Fluid Bank (IUPTFB) protocol. Samples including pancreatic ductile adenocarcinoma (n=11), mucinous cystadenoma (n=14), serous cystadenoma (n=13), IPMN high-grade/invasive (n=8), IPMN adenoma/moderate grade (n=9) and pseudocyst (n=7) were confirmed pathologically. Fluid specimens were immediately placed on ice after procurement, aliquoted for storage and stored at −80 degrees. Samples were subsequently analyzed for VEGF and those results were correlated with surgical pathologic diagnosis. Pancreatic fluid (50 μl) was analyzed by VEGF ELISA (Quantikine ELISA, R&D, Minneapolis, Minn.) which specifically detects VEGF-A. The statistical significance the data collected was determined by analysis of variance using Tukey's post test for multiple comparisons. During those evaluations the statistical significance was set at p<0.05. Results of these assays are presented in FIGS. 1, 2 and 3. FIG. 1 is a bar graph of all of the data points collected in Phase Study. While FIGS. 2 and 3 are scatter plots of respectively, all of the data and all of the data except one outlier (sample 117 which had an uncertain diagnosis). The levels of VEGF are significantly elevated in pancreatic fluid obtained from patients with serous cystadenoma compared with all other groups tested (p<0.001). Referring now to FIG. 3. The cut off for VEGF was set at 7,500 pg ml⁻¹ for the prediction of serous versus all of the other groups; the sensitivity was set at about 100% (13/12, no false negatives); the specificity was about 100% (48/48 with no false positives) this drops to 98% if data point 117 is included in the analysis.

### 2. Techniques Used to Measure the Expression of VEGF and VEGF Receptors in Tissue Samples.

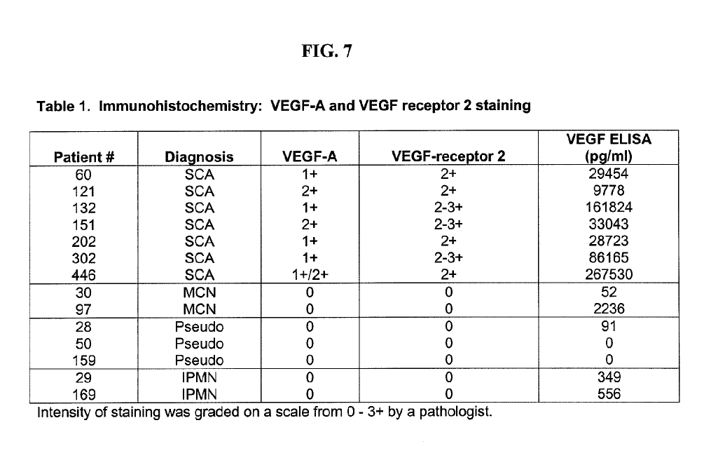

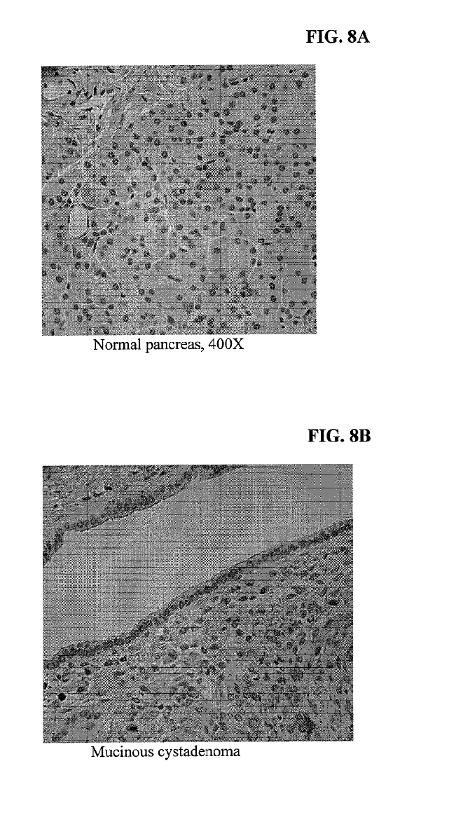

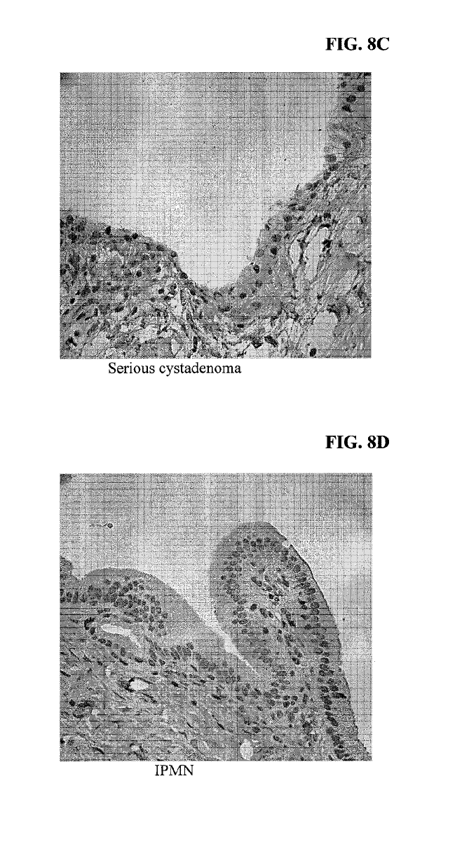

Referring now to FIGS. 8A, 8B, 8C and 8D. The expression of VEGF as well as the VEGF receptor in patients' pancreatic tissue was determined by immunohistochemistry. In these figures positive staining is indicated by differential granularity of cytoplasm [FIG. 8C, arrowheads]; a summary of these data is included in Table 1 (FIG. 7). In these studies the VEGF protein was localized to cells lining the cyst in serous cystadenoma samples, suggesting that increased VEGF protein production in these cells may lead to elevated levels of secreted VEGF in serous cystadenoma pancreatic fluid. Similarly, expression of the VEGF receptor was also localized to the cyst lining of serous cystadenoma samples. In contrast, little or no staining of VEGF or the VEGF receptor was observed in either normal pancreatic tissue or other pancreatic lesions.

### 3. Determining the Efficacy of Both PGE₂ and VEGF as Biomarkers for Pancreatic Lesions.

The sensitivity and specificity of both of these biomarkers (PGE₂ and VEGF) was determined and the data plotted, see for example, FIGS. 2, 3, 9 and 10. The level of PGE₂, was measured using the same sample stocks and methods reported on in Schmidt et al., J. Gastrointest Surg (2008) 12:243-249. Briefly, pancreatic fluid (50 μl) was analyzed by PGE₂ enzyme-linked immunosorbent assay (ELISA) (Amersham Biosciences, Piscataway, N.J.). The assay is based upon competition between unlabeled PGE₂ and a fixed quantity of peroxidase-labeled PGE₂ for binding to a PGE₂-specific antibody bound to a plate. The amount of the bound PGE₂ peroxidase is measured by the addition of the substrate. Results are expressed as pg PGE₂ per microliter.

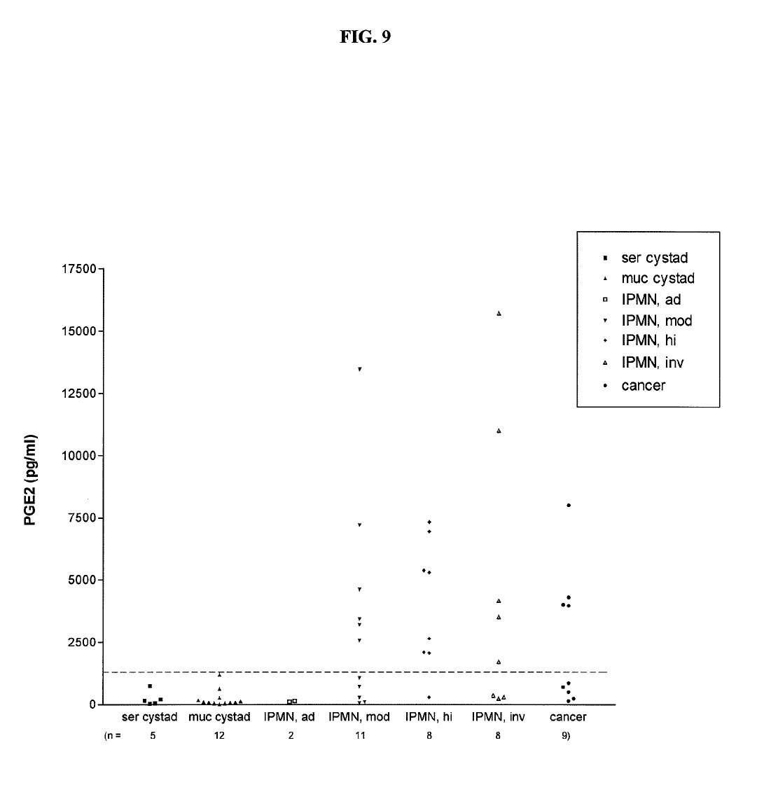

Referring now to FIG. 9. The cut-off for PGE₂ was set at about 1,250 pg ml⁻¹ for the prediction of malignant disease (IPMN high grade, invasive and pancreatic cancer); the specificity was about 64% (16/25); and the sensitivity was about 80% (24/30).

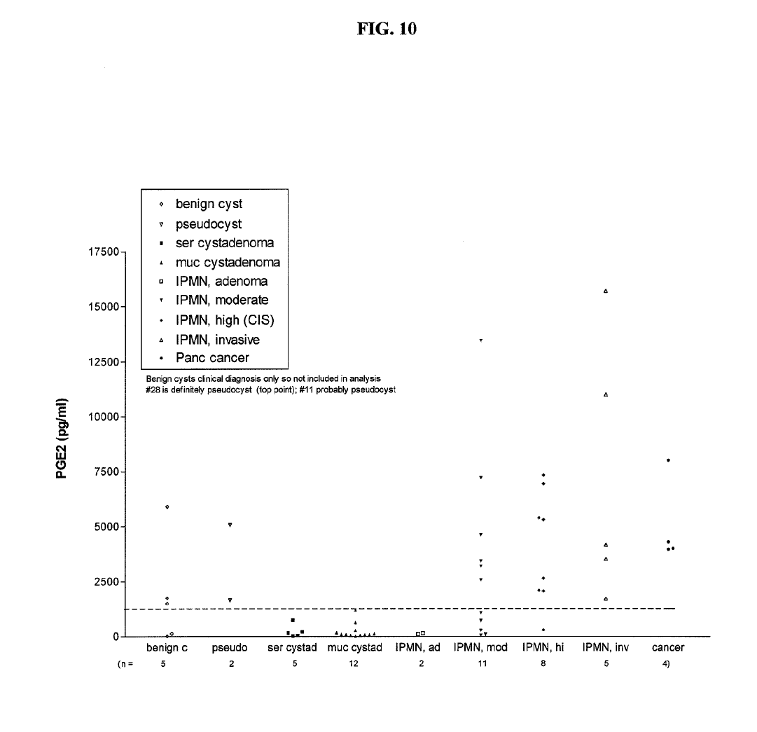

Referring now to FIG. 10. The cut-off for PGE₂ was set at about 1,250 pg ml⁻¹ (not including benign cysts) for the prediction of malignant disease (IPMN high grade, invasive and pancreatic cancer); the sensitivity was about 94% (16/17); and the specificity was about 80% (24/30) or about 75% (24/32) if data from the pseudo cysts were included. With regard to the prediction of IPMN/cancer (IPMN moderate, high, invasive and pancreatic cancer) the specificity was about 78% (22/28); and the sensitivity was either about 100% (without the inclusion of the data from the pseudocysts) and about 90% (19/21) with the inclusion of the pseudocysts.

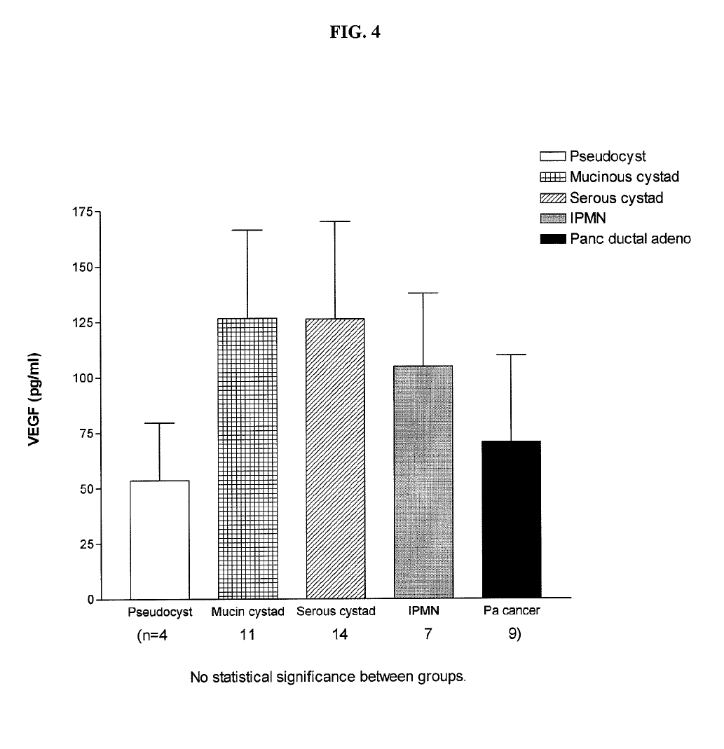

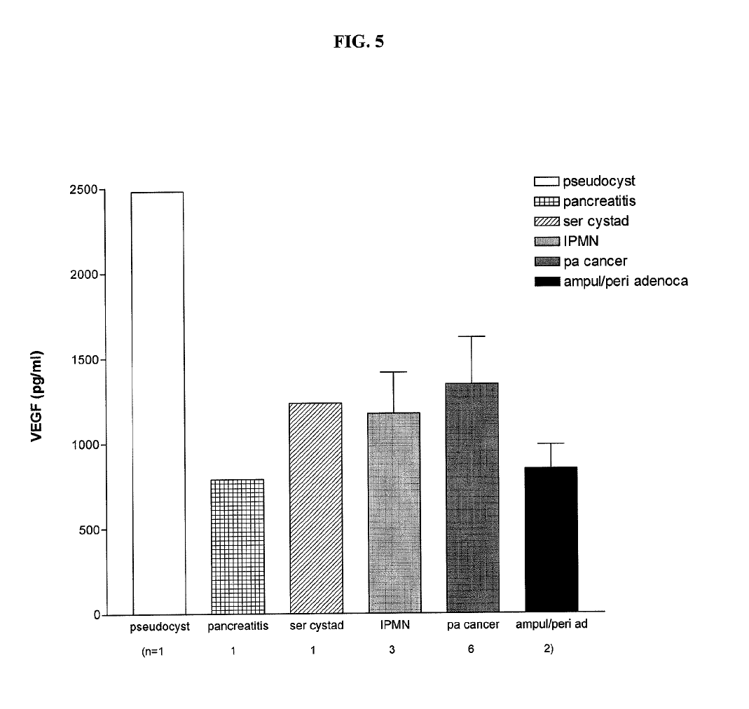

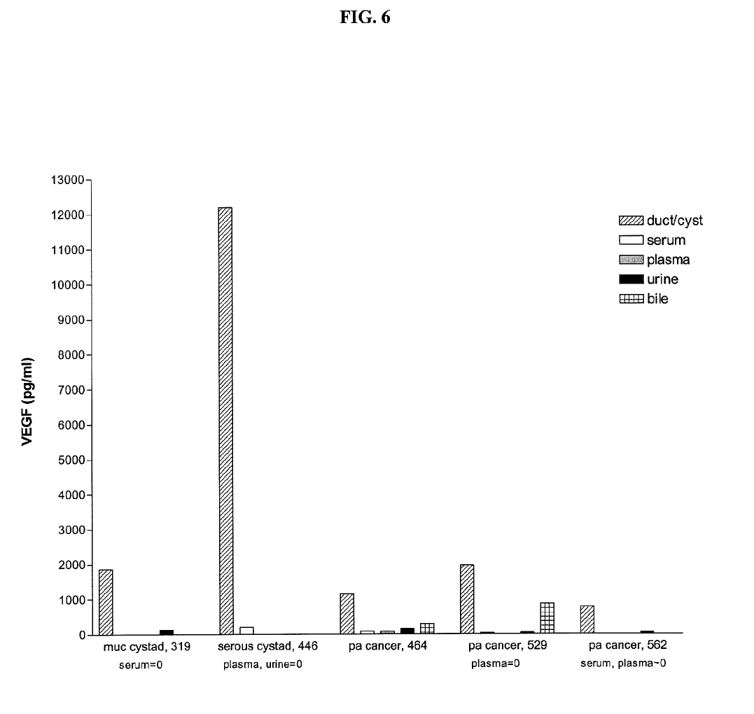

PGE₂, levels measured in samples obtained from patients without ductile obstruction, demonstrated a sensitivity of about 94% and a specificity of about 75% for predicting malignant disease (IPMN high grade/invasive and pancreatic ductal adenocarcinoma) when the cutoff for assignment set at 1250 pg ml⁻¹. PGE₂ sensitivity and specificity for predicting IPMN (moderate, high or invasive) and cancer were about 78% and about 90% respectively. VEGF provided a sensitivity of about 100% and specificity of about 98% for predicting SCA versus all other groups when the cutoff for the determination set at 7500 pg ml⁻¹. Referring now to FIGS. 4, 5 and 6. The levels of VEGF were also examined in other bodily fluids such as serum, plasma, urine and bile. In general, VEGF levels in these fluids were lower than those detected in pancreatic fluids, cyst or duct. In addition, VEGF levels in these other fluids did not differ significantly between the different pancreatic lesions and were not as predictive for pancreatic disease as were VEGF levels measured pancreatic fluid.

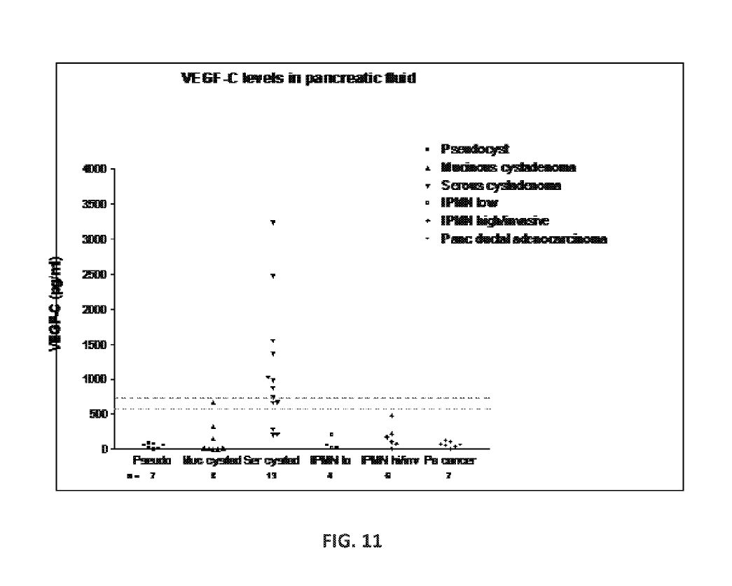

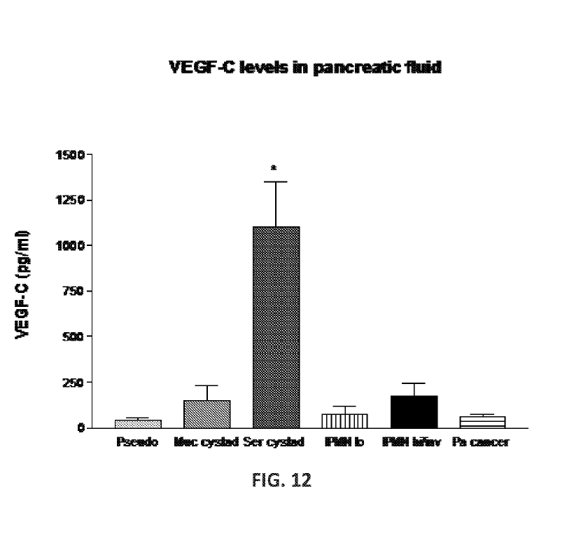

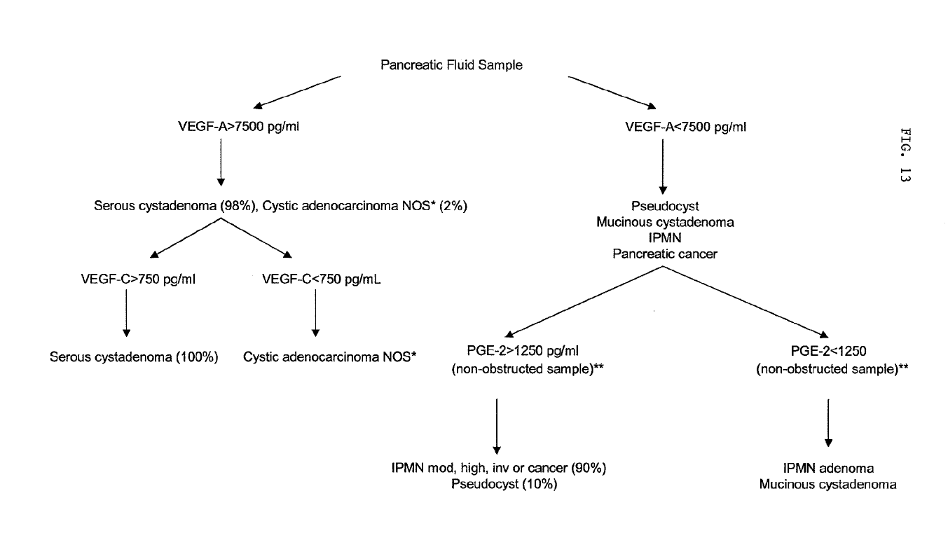

Referring now to FIGS. 11 and 12. The levels of VEGF-C was measured in pancreatic fluid recovered from patients symptomatic for various anomalies of the pancreas including pseudo cysts, mucinous cystadenocarcinoma (Muc cystads), serous cystadenoma (Ser cystad), intraductal papillary mucinous neoplasms (IMPN lo), intraductal papillary mucinous neoplasms (IPMN hi/inv) and pancreatic cancer (Pa Cancer). The VGEF-A outlier sample 117 had a VEGF-A level of about 18,391 pg ml⁻¹ and a VEGF-C value of about 220 pg ml⁻¹. By measuring both VEGF-C and VEGF-A levels in a sample and combining these values it is possible to diagnose samples representative of serous cystadenoma with, or nearly with, 100 percent specificity.

Referring now to FIG. 11, VEGF-C values (pg/m) plotted in different samples of pancreatic fluid and plotted as a function of different types of pancreatic lesions. The upper horizontal dashed line in FIG. 11 is drawn at about 750 pg ml⁻¹ of VEGF-C. When the levels of VEGF-A and VEGF-C are identified in a sample of pancreatic fluid, fluids with VEGF-C levels of about 750 pg ml⁻¹ or higher are indicative of serous cystadenoma. Still referring to FIG. 11, the lower horizontal dashed line is drawn at about 600 pg ml⁻¹ or lower. When both VEGF-C and VEGF-A are measured in a given sample all high grade cancers (carcinoma in situ) lesions and invasive cancers can be excluded if the level of VEGF-C is higher than about 600 pg ml⁻¹ and lower than about 750 pg ml⁻¹. In this data set, only one sample that had a VEGF-C value in this range was identified as being from a patient diagnosed with low grade mucinous cystadenoma.

Referring now to FIG. 12. VEGF-C levels measured in pancreatic fluids, VEGF-C (pg ml⁻¹) measured in different samples are plotted as a function of different types of pancreatic lesions.

Referring now to FIG. 13, the abbreviations used in this figure are as follows. NOS* (not otherwise specified) equivalent to stage IV (metastatic) mucinous adenocarcinoma. This particular diagnosis was based upon surgical pathology of the patient's liver and peritoneal metastases. No pancreatic resection was performed in this example. Accordingly, it is not certain whether IPMN is invasive, mucinous cystadenocarcinoma or adenocarcinoma with mucinous features. The symbol ** is used in FIG. 13 to remind the reader that a diagnosis of this nature based on levels of PGE₂ cannot be made using samples collected from a patient if the patient's main pancreatic duct is obstructed.

FIG. 13 depicts a decision tree that was constructed using data collected by measuring the levels of VEGF-A and/or VEGF-C and/or PGE₂ in a sample of pancreatic fluid assayed by using an immuno-reactive testing method. Once the levels of these compounds have been tested in various fluids and for specific pathologies a software program can be used to estimate the likelihood that a given patient is afflicted with a given type of pancreatic cyst, cancer or lesion.

Some embodiments of the invention include methods for the differential diagnosis of pancreatic lesions. It is surprising that tissue/fluid recovered from a benign lesion (i.e., serous cystadenoma) exhibits high levels of VEGF, while tissue/fluid samples taken from patients with pre-malignant or malignant pancreatic lesions do not. This relationship between VEGF expression and pancreatic disease is unexpected. Interestingly, there is one outlier in the data, a sample collected from a patient with an obvious invasive cystic cancer unambiguously diagnosed by radiographic imaging. This sample has a high level of VEGF-A, on the order of about 18,391 pg ml⁻¹ and a VEGF-C level of only about 220 pg ml⁻¹ Based this sample's low VEGF-C level it was not collected from a patient with serous cystadenomas. This sample is likely to be from a patient afflicted with IPMN an invasive form of pancreatic cancer. In any event, patients with this particular form of pancreatic cancer can be diagnosed without the aid of a pancreatic fluid based test, because when a patient presents with an invasive cancer this advanced preoperative imaging is sufficient for ruling out serous cystadenoma and establishing a diagnosis of invasive cancer. For patients with advanced pancreatic cancer readily identified by radiographic imaging, the opportunity for discriminating between the benign form and the malignant form and/or early intervention will have passed, so a method for identification/differentiation of serous cystadenoma from other more worrisome pancreatic conditions may have no meaningful effect on treatment options available to such patients.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

We claim:

1. A method of diagnosing pancreatic lesions, comprising the steps of:
measuring the level of VEGF-A in the sample by contacting the sample with at least one compound that preferentially binds to VEGF-A, wherein the sample is from a patient;
determining that the patient, has at least one pancreatic pathology selected from the group consisting of Serous cystadenoma and Cystic adenocarcinoma if the level of VEGF-A measured in the sample is greater than about 7,500 pg ml⁻¹.

2. The method according to claim 1, further including the steps of:
measuring the level of VEGF-C in the sample by contacting the sample with at least one compound that preferentially binds to VEGF-C.

3. The method according to claim 2, further including the step of:
determining that the patient has serous cystadenoma if the level of VEGF-C in the sample is greater than about 750 pg ml⁻¹.

4. The method according to claim 2, further including the step of:
determining that the patient has cystic adenocarcinoma if the level of VEGF-C in the sample is less than about 750 pg ml⁻¹.

5. The method according to claim 1, wherein the compound that binds to VEGF-A is an antibody.

6. The method according to claim 1 further including the step of:
collecting the sample from the patient.

7. The method according to claim 2, wherein the compound that binds to VEGF-A is an antibody.

8. The method according to claim 2 wherein the compound that binds to VEGF-C is a monoclonal anitbody.

9. A kit for diagnosing pancreatic lesions, comprising:
a first compound that binds to VEGF-A;
a second compound that binds to VEGF-C; and
at least one buffer.

10. The kit for diagnosing pancreatic lesions according to claim 9, wherein the first compound that binds to VEGF-A is an antibody to VEGF-A and the second compound that binds to VEGF-C is an antibody to VEGF-C.

11. The kit for diagnosing pancreatic lesions according to claim 9, wherein the first compound that binds to VEGF-A is a monoclonal antibody raised to VEGF-A, and the second compound that binds to VEGF-C is monoclonal antibody raised to VEGF-C.

12. A method of diagnosing pancreatic lesions, comprising the steps of:
obtaining a sample of pancreatic fluid wherein the sample was collected from a patient, wherein the patient has at least one pancreatic lesion;
measuring the level of VEGF-A in the sample by contacting the sample with at least one compound that preferentially binds to VEGF-A;
determining that the patient has at least one pancreatic pathology selected from the group consisting of; pseudocyst, munincous cystadenoma, IPMN pancreatic cancer if the level of VEGF-A measured in the sample is less than about 7,500 pg ml⁻¹.

13. The method according to claim 12, further including the steps of:
measuring the level of PGE₂ in the sample.

14. The method according to claim 13, further including the step of:
determining that the patient has at least a nine out of 10 chance of having at one pancreatic pathology selected from the group consisting of IPMN mod. high, invasive pancreatic cancer and about a one out of 10 chance of having a pseudocyst if the level of PGE₂ in the sample is greater than about 1,250 pg ml⁻¹, and the patient's main pancreatic duct is not obstructed.

15. The method according to claim 13, further including the step of:
determining that the patient has IPMN adenoma or mucinous cystadenome if the level of PGE₂ in the sample is less than about 1,250 pg ml⁻¹ and the patient's main pancreatic duct is not obstructed.

16. The method according to claim 12, wherein the compound that binds to VEGF-A is an antibody.

17. The method according to claim 12 wherein the compound that binds to VEGF-A is monoclonal antibody raised against VEGF-A.

18. A kit for diagnosing pancreatic lesions, comprising:
a first compound that binds to VEGF-A;
a reagent for measuring the amount of PGE₂ in the sample.

19. The kit for diagnosing pancreatic lesions according to claim 18, wherein the first compound that binds to VEGF-A is an antibody to VEGF-A.

20. The kit for diagnosing pancreatic lesions according to claim 18, wherein the first compound that binds to VEGF-A is a monoclonal antibody to VEGF-A.

21. The kit for diagnosing pancreatic lesions according to claim 18, wherein the kit further includes at least one buffer.
